(19) United States
(12) Patent Application Publication
(10) Pub. No.: US 2022/0313478 A1
(43) Pub. Date: Oct. 6, 2022

(54) Fluid Delivery Lines of Systems, Pads, and Methods for Targeted Temperature Management

(71) Applicant: C. R. Bard, Inc., Franklin Lakes, NJ (US)

(72) Inventors: Gabriel A. Johnston, Broomfield, CO (US); Mengjia Yi, Westminster, CO (US); Jesse Smith, Broomfield, CO (US); Niharika Pathare, Thane (IN); Harry D. Turner, Cambridge (GB); Andrew R. Taylor, Cambridge (GB)

(21) Appl. No.: 17/709,019

(22) Filed: Mar. 30, 2022

Related U.S. Application Data
(60) Provisional application No. 63/168,146, filed on Mar. 30, 2021.

(51) Int. Cl. A61F 7/00

(52) U.S. Cl. CPC A61F 7/0085; A61F 2007/0056; A61F 2007/0093; A61F 2007/0244

(57) ABSTRACT

Disclosed are fluid delivery lines (“FDLs”) of systems, pads, and methods for targeted temperature management. A system can include a control module, a primary FDL, a secondary FDL, and one or more pads for placement on one or more portions of a patient's body, respectively. The primary FDL can be configured to convey a temperature-controlled fluid as a supply fluid from a hydraulic system of the control module and convey a return fluid back to the hydraulic system. Each pad of the one-or-more pads can include a multilayered pad body including a conduit layer configured to convey the supply fluid therethrough. The secondary FDL can be configured to convey the supply fluid from the primary FDL and convey the return fluid back to the primary FDL. The secondary FDL can include a pair of piercing tips configured to pierce the primary tubing for fluidly connecting the primary and secondary FDLs.

### PRIORITY

This application claims the benefit of priority to U.S. Provisional Patent Application No. 63/168,146, filed Mar. 30, 2021, which is incorporated by reference in its entirety into this application.

### BACKGROUND

Targeted temperature management (“TTM”) is a treatment for maintaining a therapeutic body temperature (e.g., hypothermia) in a patient for a period of time to improve the patient's outcome in any of a number of different medical situations. Any of a number of current systems for TTM use a number of fluid delivery lines to convey temperature-controlled fluid from a control module to one or more pads placed on one or more portions of the patient's body. With the number fluid delivery lines and connectors thereof that can be required in a given medical situation, it can be difficult to fluidly connect the number of fluid delivery lines together quickly and accurately, particularly for an inexperienced clinician under a time constraint. What is needed are fluid delivery lines that can be quickly and accurately connected even by clinicians inexperienced with the current systems for TTM.

Disclosed herein are fluid delivery lines of systems, pads, and methods thereof for TTM.

### SUMMARY

Disclosed herein is a system for TTM including, in some embodiments, a control module, a primary fluid delivery line (“FDL”), a secondary FDL, and one or more pads. The control module includes a hydraulic system configured to provide a temperature-controlled fluid. The primary FDL includes primary tubing configured to convey the temperature-controlled fluid as a supply fluid from the hydraulic system. The primary tubing is also configured convey a return fluid back to the hydraulic system. The one-or-more pads are configured for placement on one or more portions of a patient's body, respectively. Each pad of the one-or-more pads includes a multilayered pad body and the secondary FDL. The pad body includes a conduit layer, which, in turn, includes one or more conduits configured to convey the supply fluid through the conduit layer. The secondary FDL includes secondary tubing configured to convey the supply fluid from the primary FDL. The secondary tubing is also configured convey the return fluid back to the primary FDL. The secondary FDL includes a pair of piercing tips configured to pierce the primary tubing and, thereby, fluidly connect the secondary FDL tubing to the primary FDL.

In some embodiments, the primary tubing is self-sealing elastomeric tubing.

In some embodiments, the primary tubing includes a closed end and a connector end. The connector end of the primary tubing includes a primary FDL connector configured to fluidly connect to a control-module connector of the control module.

In some embodiments, the primary tubing includes a longitudinal septum short of the closed end of the primary tubing that forms a folded lumen in the primary tubing. The folded lumen is configured to convey the supply fluid from an inlet of the primary FDL connector to the closed end of the primary tubing. The folded lumen is also configured to convey the return fluid from the closed end to an outlet of the primary FDL connector of the primary tubing.

In some embodiments, the primary tubing includes a pair of opposing connector ends respectively including a pair of primary FDL connectors. The pair of primary FDL connectors are configured to fluidly connect to a pair of control-module connectors of the control module.

In some embodiments, the primary tubing includes a regular lumen in the primary tubing. The regular lumen is configured to convey the supply fluid from an inlet of a primary inlet connector of the pair of primary FDL connectors. The regular lumen is also configured to convey the return fluid to an outlet of a primary outlet connector of the pair of primary FDL connectors.

In some embodiments, the pad body further includes an impermeable film over the conduit layer and a thermally conductive adhesive layer over the impermeable film. The impermeable film is configured to retain the supply fluid in the conduit layer. The adhesive layer is configured for placement on a portion of a patient's body.

In some embodiments, the adhesive layer includes a hydrogel. The hydrogel is selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, each pad of the one-or-more pads further includes a backing over the adhesive layer in a ready-to-use state of the pad. The backing is configured to maintain integrity of at least the adhesive layer prior to use of the pad.

In some embodiments, each pad of the one-or-more pads further includes a pad inlet and a pad outlet. The pad inlet is configured for charging the conduit layer with the supply fluid. The pad outlet is configured for discharging the return fluid from the conduit layer.

In some embodiments, the secondary FDL is split at a pad-connecting end of the secondary FDL. The pad-connecting end includes a pair of secondary FDL connectors. A secondary inlet connector of the pair of secondary FDL connectors is configured to fluidly connect to the pad outlet. A secondary outlet connector of the pair of secondary FDL connectors is configured to fluidly connect to the pad inlet.

In some embodiments, the secondary FDL is split at a primary FDL-connecting end of the secondary FDL. The primary FDL-connecting end includes the pair of piercing tips. A secondary inlet piercing tip of the pair of piercing tips is configured to fluidly connect to the primary FDL. A secondary outlet piercing tip of the pair of piercing tips is configured to fluidly connect to the primary FDL in series with the secondary inlet piercing tip.

In some embodiments, the hydraulic system further includes a chiller evaporator, a heater, a hydraulic-system outlet, and a hydraulic-system inlet. The chiller evaporator is configured for fluid cooling. The heater is configured for fluid heating. Together, the chiller evaporator and the heater are configured to provide the temperature-controlled fluid. The hydraulic-system outlet is configured for discharging the supply fluid from the hydraulic system. The hydraulic-system inlet is configured for charging the hydraulic system with the return fluid to continue to produce the temperature-controlled fluid.

In some embodiments, the control module further includes one or more processors, primary memory, and instructions stored in the primary memory. The instructions are configured to instantiate one or more processes for TTM with the control module. At least one process of the one-or-more processes is a fluid delivery-adjustment process. The fluid delivery-adjustment process is configured for determining and adjusting parameters of fluid delivery in accordance with a number of FDL-connected pads of the one-or-more pads.

Also disclosed herein is a method of a system for TTM including, in some embodiments, a piercing step, a pad-placing step, a fluid-conveying step, and a fluid-circulating step. The piercing step includes piercing primary tubing of a primary FDL with a pair of piercing tips of a secondary FDL to fluidly connect the secondary FDL tubing to the primary FDL. The pad-placing step includes placing a pad on a portion of a patient's body with a thermally conductive adhesive layer of a multilayered pad body of the pad in contact with skin of the portion of the patient's body. The fluid-conveying step includes conveying a temperature-controlled fluid as a supply fluid to the pad. The supply fluid provided by a hydraulic system of a control module by way of the primary and secondary FDLs. The fluid-circulating step includes circulating the supply fluid through a conduit layer of the pad body to cool or warm the portion of the patient's body as needed in accordance with TTM.

In some embodiments, the method further includes a control module-connecting step. The control module-connecting step includes fluidly connecting a primary FDL connector of a connector end of the primary FDL to a control-module connector of the control module. The primary tubing includes a longitudinal septum short of a closed end of the primary FDL that forms a folded lumen in the primary tubing. The folded lumen is configured for conveying the supply fluid from an inlet of the primary FDL connector. The folded lumen is also configured for conveying a return fluid to an outlet of the primary FDL connector.

In some embodiments, the method further includes an alternative control module-connecting step. The alternative control module-connecting step includes fluidly connecting a pair of primary FDL connectors respectively at opposing connector ends of the primary FDL to a pair of control-module connectors of the control module. The primary tubing includes a regular lumen. The regular lumen is configured for conveying the supply fluid from an inlet of a primary inlet connector of the pair of primary FDL connectors. The regular lumen is also configured for conveying a return fluid to an outlet of a primary outlet connector of the pair of primary FDL connectors.

In some embodiments, the method further includes a pad-connecting step. The pad-connecting step includes fluidly connecting a secondary outlet connector of a pair of secondary FDL connectors at a split pad-connecting end of the secondary FDL to a pad outlet of the pad. The pad-connecting step also includes fluidly connecting a secondary inlet connector of the pair of secondary FDL connectors to a pad inlet of the pad.

In some embodiments, the method further includes a backing-removal step. The backing-removal step includes removing a backing of the pad to reveal the adhesive layer before the pad-placing step. The backing is configured to maintain integrity of at least the adhesive layer prior to using the pad.

In some embodiments, the fluid-circulating step includes transferring heat between the supply fluid and the portion of the patient's body by thermal conduction through the adhesive layer. The adhesive layer includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, the fluid-circulating step includes circulating a cool fluid through the conduit layer to bring the patient into hypothermia.

In some embodiments, the fluid-circulating step circulating a warm fluid through the conduit layer to bring the patient into normothermia.

In some embodiments, the method further includes a disconnecting step. The disconnecting step includes removing the pair of piercing tips of the secondary FDL from the primary tubing of the primary FDL to disconnect the secondary FDL tubing from the primary FDL. The primary tubing is self-sealing elastomeric tubing configured to prevent leaking when removing the pair of piercing tips of the secondary FDL from the primary tubing of the primary FDL.

Also disclosed herein is a pad for TTM including, in some embodiments, a multilayered pad body and a secondary FDL. The pad body includes a conduit layer, an impermeable film over the conduit layer, and a thermally conductive adhesive layer over the impermeable film. The conduit layer includes one or more conduits configured to convey a temperature-controlled fluid as a supply fluid from a hydraulic system of a control module as well as convey a return fluid back to the hydraulic system. The impermeable film is configured to retain the supply fluid in the conduit layer. The adhesive layer is configured for placement on a portion of a patient's body. The secondary FDL includes secondary tubing configured to convey the supply fluid from a primary FDL fluidly connected to the hydraulic system as well as convey the return fluid back to the primary FDL. The secondary FDL includes a secondary FDL plug-and-receptacle connector configured to fluidly connect to a complementary primary FDL connector of the primary FDL or an intervening secondary FDL plug-and-receptacle connector of an intervening pad between the secondary FDL plug-and-receptacle connector and the primary FDL connector.

In some embodiments, the secondary FDL plug-and-receptacle connector includes up to two protrusions extending from a plug portion of the plug-and-receptacle connector. In addition, the secondary FDL plug-and-receptacle connector includes up to two openings in a receptacle portion of the plug-and-receptacle connector.

In some embodiments, each protrusion of the up-to-two protrusions extends from the plug portion of the plug-and-receptacle connector in a same direction as another protrusion of the up-to-two protrusions.

In some embodiments, each protrusion of the up-to-two protrusions extends from the plug portion of the plug-and-receptacle connector in an opposite direction from another protrusion of the up-to-two protrusions.

In some embodiments, each protrusion of the up-to-two protrusions extends from the plug portion of the plug-and-receptacle connector in a direction of fluid flow for the temperature-controlled fluid.

In some embodiments, each protrusion of the up-to-two protrusions extending from the plug portion of the plug-and-receptacle connector includes at least one circumferential groove including an ‘O’-ring disposed therein. With the ‘O’-ring, the protrusion is configured to seal a fluid connection established between the protrusion and a corresponding opening in a receptacle portion of the plug-and-receptacle connector.

In some embodiments, each opening of the up-to-two openings in the receptacle portion of the plug-and-receptacle connector includes a valve disposed therein. With the valve, the opening is configured to seal off the opening when a corresponding protrusion extending from the plug portion of the plug-and-receptacle connector does not extend through the valve.

In some embodiments, the secondary FDL plug-and-receptacle connector includes a pluggable cap extending from a plug portion of the plug-and-receptacle connector as well as a socket in a receptacle portion of the plug-and-receptacle connector. The cap is configured to insert into another socket in the receptacle portion of another plug-and-receptacle connector, and the socket configured to accept insertion of another cap in the plug portion of yet another plug-and-receptacle connector.

Also disclosed herein is a system for TTM including, in some embodiments, a primary FDL and one or more pads. The primary FDL includes primary tubing configured to convey a temperature-controlled fluid as a supply fluid from a hydraulic system as well as convey a return fluid back to the hydraulic system. The primary FDL includes a plurality of primary FDL connectors along a length of the primary tubing. The one-or-more pads are configured for placement on one or more portions of a patient's body, respectively. Each pad of the one-or-more pads includes a multilayered pad body having a conduit layer including one or more conduits configured to convey the supply fluid through the conduit layer. In addition, each pad of the one-or-more pads includes a secondary FDL having secondary tubing configured to convey the supply fluid from the primary FDL as well as convey the return fluid back to the primary FDL. The secondary FDL includes a secondary FDL connector configured to fluidly connect to any primary FDL connector of the plurality of primary FDL connectors along the length of the primary tubing.

In some embodiments, the plurality of primary FDL connectors are regularly spaced along the length of the primary tubing.

In some embodiments, each primary FDL connector of the plurality of primary FDL connectors is configured to fluidly connect up to two secondary FDL connectors respectively of two pads.

In some embodiments, each primary FDL connector of the plurality of primary FDL connectors includes a pair of oppositely oriented plug portions of the primary FDL connector. Each plug portion of the pair plug portions includes a pair of protrusions extending therefrom.

In some embodiments, the secondary FDL connector includes a pair of openings in a receptacle portion of the secondary FDL connector. The pair of openings are configured to form fluid connections with the pair of protrusions.

These and other features of the concepts provided herein will become more apparent to those of skill in the art in view of the accompanying drawings and following description, which describe particular embodiments of such concepts in greater detail.

### DRAWINGS

FIG. 1 illustrates a TTM system in accordance with some embodiments.

FIG. 2A illustrates a primary FDL including a folded lumen configured to convey fluid to and from a control module of the TTM system in accordance with some embodiments.

FIG. 2B illustrates a primary FDL including a regular lumen configured to convey fluid to and from the control module of the TTM system in accordance with some embodiments.

FIG. 3A illustrates left and right TTM pads for a torso of a patient in accordance with some embodiments.

FIG. 3B illustrates left and right TTM pads for legs of a patient in accordance with some embodiments.

FIG. 4 illustrates TTM pads including secondary FDLs with secondary FDL plug-and-receptacle connectors configured to fluidly connect to a complementary primary FDL connector of the primary FDL in accordance with some embodiments.

FIG. 5 illustrates the TTM pads including the secondary FDLs with alternative secondary FDL plug-and-receptacle connectors configured to fluidly connect to a complementary primary FDL connector of the primary FDL in accordance with some embodiments.

FIG. 6 illustrates a pad including a secondary FDL with a secondary FDL receptacle connector configured to fluidly connect to any primary FDL connector of a plurality of primary FDL connectors along a length of primary tubing of the primary FDL in accordance with some embodiments.

FIG. 7 illustrates a longitudinal cross section of a primary FDL connector of the plurality of primary FDL connectors of the primary FDL of FIG. 6 in accordance with some embodiments.

FIG. 8 illustrates a multilayered pad body of a TTM pad in accordance with some embodiments.

FIG. 9 illustrates a hydraulic system of a control module in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth above, any of a number of current systems for TTM use a number of fluid delivery lines to convey temperature-controlled fluid from a control module to one or more pads placed on one or more portions of the patient's body. With the number fluid delivery lines and connectors thereof that can be required in a given medical situation, it can be difficult to fluidly connect the number of fluid delivery lines together quickly and accurately, particularly for an inexperienced clinician under a time constraint. What is needed are fluid delivery lines that can be quickly and accurately connected even by clinicians inexperienced with the current systems for TTM.

Disclosed herein are fluid delivery lines of systems, pads, and methods thereof for TTM.

### TTM Systems

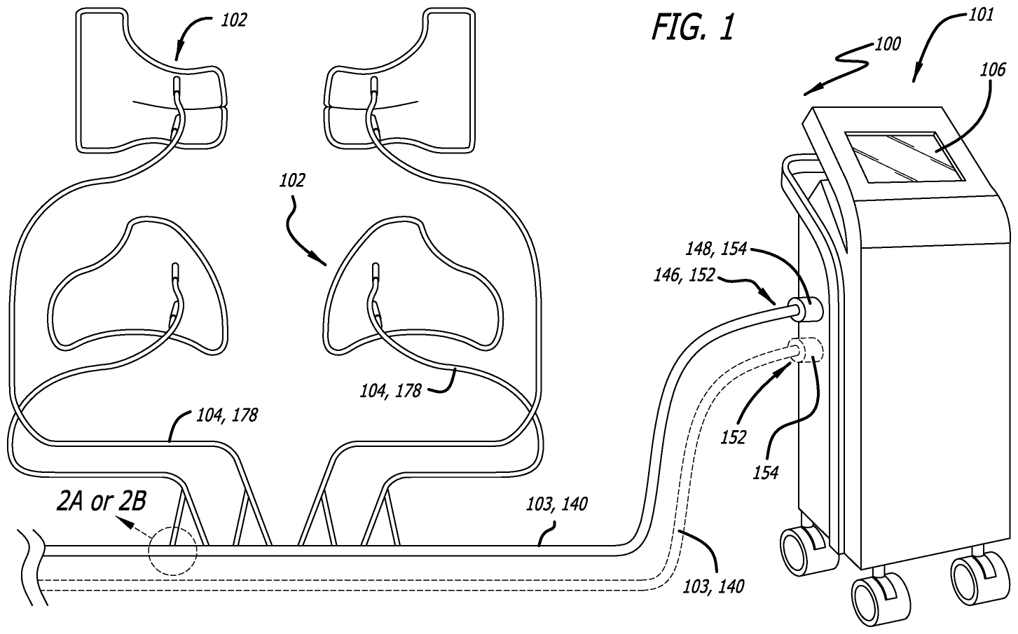

FIG. 1 illustrates a TTM system 100 in accordance with some embodiments.

As shown, the system 100 can include a control module 101, one or more TTM pads 102 such as those set forth below, a primary FDL 103, and one or more secondary FDLs 104 corresponding in number to the one-or-more TTM pads 102. Description for the control module 101 is set forth immediately below. Description for the one-or-more pads 102 is set forth in the following section.

The control module 101 can include a console 106 with an integrated display screen configured as a touchscreen for operating the control module 101. The console 106 can include one or more processors, primary and secondary memory, and instructions stored in the primary memory configured to instantiate one or more processes for TTM with the control module 101. At least one process of the one-or-more processes can be a fluid delivery-adjustment process. The fluid delivery-adjustment process can be configured for determining and adjusting parameters of fluid delivery in accordance with a number of FDL-connected pads of the one-or-more pads 102. For example, a drop in pressure upon adding each additional pad of the one-or-more pads 102 is compensated by an increase in pressure in the primary FDL 103 by way of the fluid delivery-adjustment process.

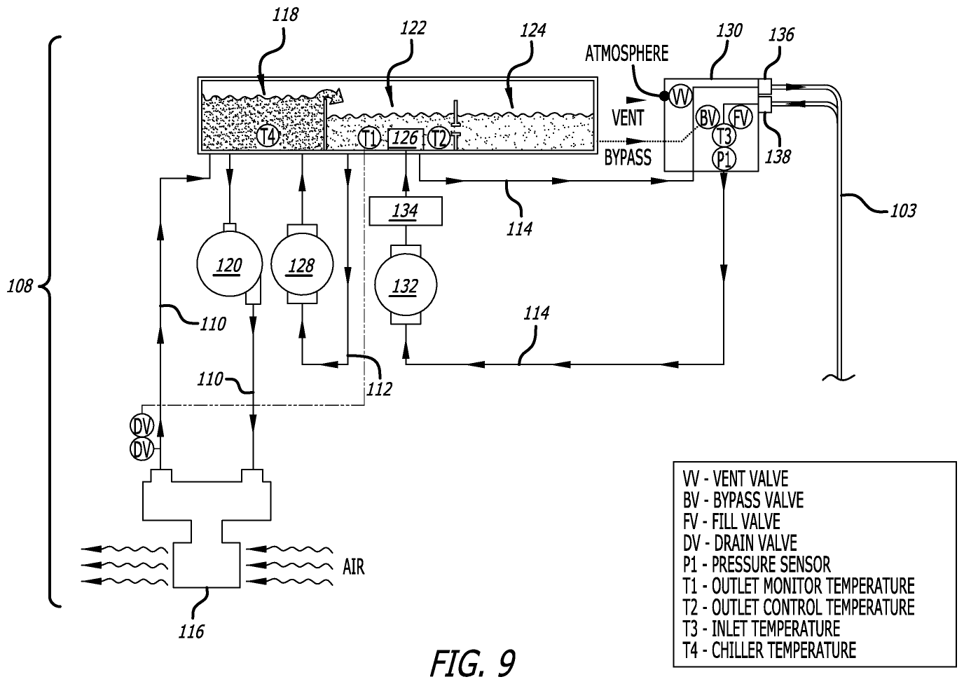

FIG. 9 illustrates a hydraulic system 108 of the control module 101 in accordance with some embodiments.

The control module 101 can also include the hydraulic system 108, which can include a chiller circuit 110, a mixing circuit 112, and a circulating circuit 114 for providing a temperature-controlled fluid.

The chiller circuit 110 can be configured for cooling a fluid (e.g., water, ethylene glycol, a combination of water and ethylene glycol, etc.) to produce a cooled fluid, which cooled fluid, in turn, can be for mixing with the mixed fluid in the mixing tank 122 set forth below to produce a supply fluid for TTM. The chiller circuit 110 can include a chiller evaporator 116 configured for the cooling of the fluid passing therethrough. The fluid for the cooling by the chiller evaporator 116 is provided by a chiller tank 118 using a chiller pump 120 of the chiller circuit 110.

The mixing circuit 112 can be configured for mixing spillover of the cooled fluid from the chiller tank 118 with a mixed fluid in a mixing tank 122 of the mixing circuit 112. The mixing circuit 112 can include a heater 126 in the mixing tank 122 configured for heating the mixed fluid to produce a heated fluid if needed for mixing with the cooled fluid to provide a supply tank 124 of the circulating circuit 114 with the supply fluid of a desired temperature for TTM. The mixing circuit 112 can include a mixing pump 128 configured to pump the fluid from the mixing tank 122 into the chiller tank 118 for producing the cooled fluid as well as the spillover of the cooled fluid for the mixing tank 122.

The circulating circuit 114 can be configured for circulating the supply fluid for TTM, which includes circulating the supply fluid provided by a manifold 130 through the one-or-more pads 102 using a circulation pump 132 directly or indirectly governed by a flow meter 134 of the circulating circuit 114. The manifold 130 can include an outlet 136 configured for discharging the supply fluid (e.g., a cooled fluid or a warmed fluid as indicated) from the hydraulic system 108 and an inlet 138 configured for charging the hydraulic system 108 with return fluid from the one-or-more pads 102 to continue to produce the supply fluid.

The primary FDL 103 can include primary tubing 140 configured to convey the supply fluid from the hydraulic system 108 when fluidly connected thereto. Likewise, the primary tubing 140 is configured convey the return fluid back to the hydraulic system 108 when fluidly connected thereto.

The primary tubing 140 can be self-sealing elastomeric tubing configured to resist leaking after being punctured such as when fluidly connected to the one-or-more secondary FDLs 104 or subsequently disconnected from the one-or-more secondary FDLs 104.

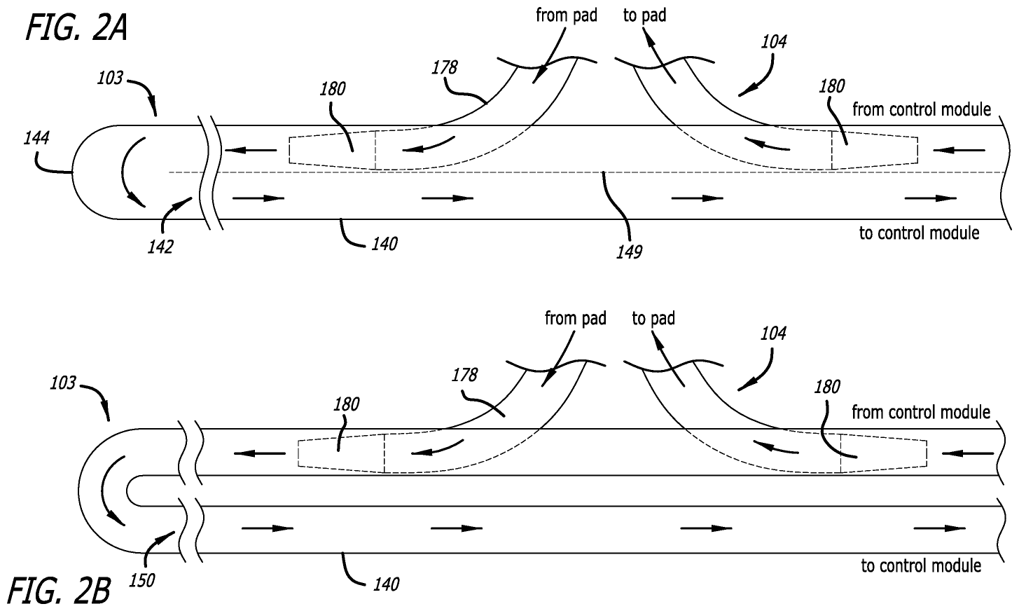

FIG. 2A illustrates the primary FDL 103 including a folded lumen 142 configured to convey the supply fluid from the control module 101 and convey the return fluid to the control module 101 in accordance with some embodiments.

As shown, the primary tubing 140 of the primary FDL 103 of FIG. 2A can include a closed end 144 opposite a connector end 146. The connector end 146 of the primary tubing 140 can include a primary FDL connector 148. The primary FDL connector 148 can be configured to connect to a control-module connector (not shown) of the control module 101.

The primary tubing 140 of the primary FDL 103 of FIG. 2A can also include a longitudinal septum 149 short of the closed end 144 of the primary tubing 140 that forms the folded lumen 142 in the primary tubing 140. The folded lumen 142 can be configured to convey the supply fluid from an inlet of the primary FDL connector 148 to the closed end 144 of the primary tubing 140. The folded lumen 142 can also configured to convey the return fluid from the closed end 144 to an outlet of the primary FDL connector 148 of the primary tubing 140.

FIG. 2B illustrates the primary FDL 103 including a regular lumen 150 configured to convey the supply fluid from the control module 101 and convey the return fluid to the control module 101 in accordance with some embodiments.

As shown, the primary tubing 140 of the primary FDL 103 of FIG. 2B can include a pair of opposing connector ends 152 respectively including a pair of primary FDL connectors 154. The pair of primary FDL connectors 154 can be configured to fluidly connect to a pair of control-module connectors (not shown) of the control module 101.

The primary tubing 140 of the primary FDL 103 of FIG. 2B can also include the regular lumen 150 in the primary tubing 140. The regular lumen 150 can be configured to convey the supply fluid from an inlet of a primary inlet connector of the pair of primary FDL connectors 154. The regular lumen 150 is also configured to convey the return fluid to an outlet of a primary outlet connector of the pair of primary FDL connectors 154. Notably, the regular lumen 150 is “regular” in that it is not folded like the folded lumen 142.

Being as the one-or-more secondary FDLs 104 correspond with the one-or-more pads 102, description for the one-or-more secondary FDLs 104 is set forth in the following section.

### TTM Pads

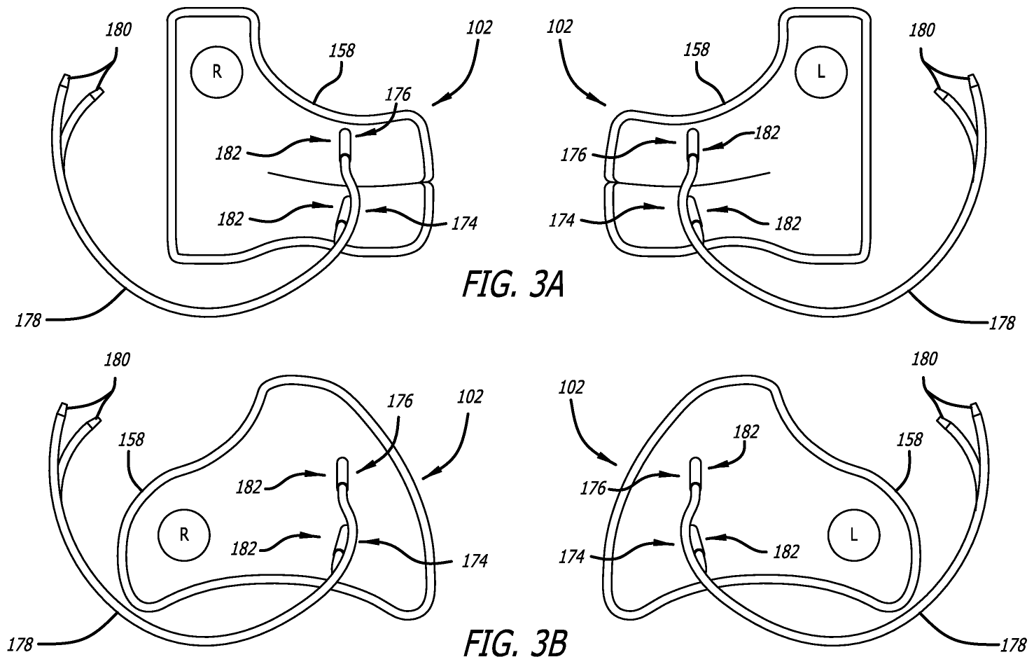

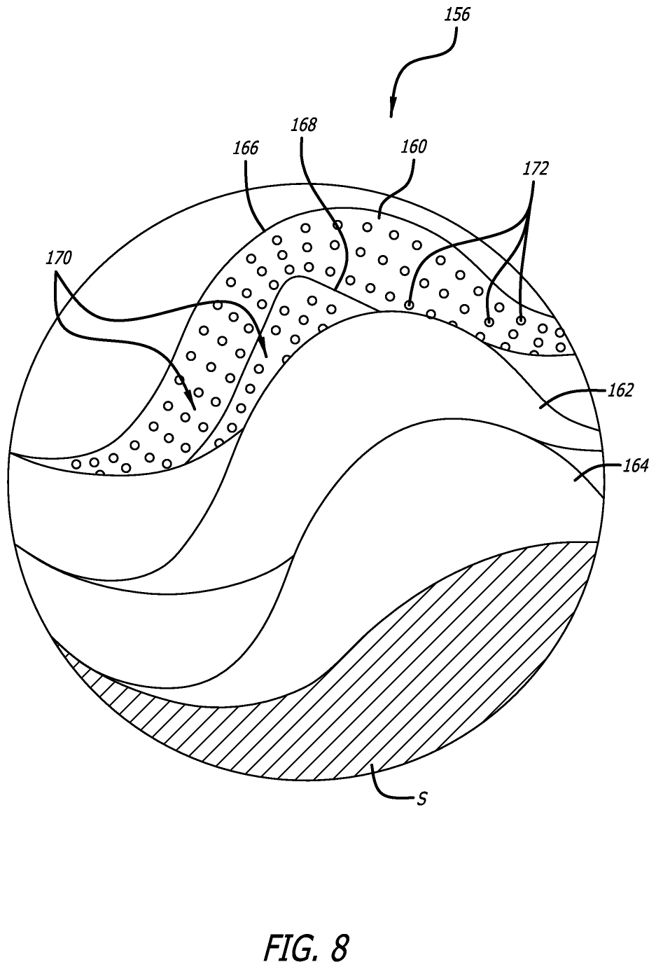

FIGS. 3A and 3B illustrate left and right pads of the one-or-more pads 102 respectively for a torso and legs of a patient in accordance with some embodiments. However, the one-or-more pads can be configured for placement on any one or more portions of a patient's body, respectively, not just the torso or legs. FIG. 8 illustrates a multilayered pad body 156 of a pad of the-one-or-more pads 102 in accordance with some embodiments.

A pad of the one-or-more pads 102 can include the pad body 156 and a backing 158 over the pad body 156.

The pad body 156 can include a conduit layer 160, an impermeable film 162 over the conduit layer 160, and a thermally conductive adhesive layer 164 over the impermeable film 162.

The conduit layer 160 includes a perimetrical wall 166 and one or more inner walls 168 extending from the conduit layer 160 toward the impermeable film 162. Together with the impermeable film 162, the perimetrical wall 166 and the one-or-more inner walls 168 form one or more conduits 170 configured to convey the supply fluid through the conduit layer 160.

The conduit layer 160 can include a plurality of protrusions 172 extending from the conduit layer 160 toward the impermeable film 162. The protrusions 172 are configured to promote even flow of the supply fluid when the supply fluid is conveyed through the conduit layer 160.

The conduit layer 160 can be of an insulating foam. The insulating foam can be configured to prevent heat loss into an ambient environment.

The impermeable film 162 can be configured to retain the supply fluid in the conduit layer 160 when the supply fluid is conveyed through the conduit layer 160. In addition, the impermeable film 162 can be configured to allow efficient energy transfer between the conduit layer 160 and the adhesive layer 164.

The adhesive layer 164 can be configured for placement on skin S (see FIG. 8) of a portion (e.g., torso, leg, etc.) of a patient's body for direct thermal conduction through the adhesive layer 164. While the adhesive layer 164 can be configured to conformably adhere to the portion of the patient's body for better thermal conduction, adherence of the adhesive layer 164 to the portion of the patient's body can be optimized to avoid irritating or wounding the portion of the patient's body upon removal of the pad.

The adhesive layer 164 can include a hydrogel or hydrogel matrix. The hydrogel can be selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

The backing 158 can be over the adhesive layer 164 in a ready-to-use state of the pad. The backing 158 is configured to maintain integrity of at least the adhesive layer 164 prior to use of the pad.

In addition to the foregoing, the pad can include an inlet 174 and an outlet 176. The inlet 174 is configured for charging the conduit layer 160 with the supply fluid, while the outlet 176 is configured for discharging the return fluid from the conduit layer 160.

A pad of the one-or-more pads 102 can include a secondary FDL of the one-or-more secondary FDLs 104. For example, the secondary FDL can be pre-connected to the pad as sold.

The secondary FDL can include secondary tubing 178 configured to convey the supply fluid from the primary FDL 103. Likewise, the secondary tubing 178 is configured to convey the return fluid back to the primary FDL 103.

The secondary FDL can include a pair of piercing tips 180 configured to pierce the primary tubing 140 and, thereby, fluidly connect the secondary FDL to the primary FDL 103. Being able to fluidly connect the one-or-more secondary FDLs 104 to the primary FDL 103 in such a way is advantageous in that it is akin to establishing a percutaneous puncture, insertion of a catheter into an insertion site, etc., which is a familiar practice to clinicians. The one-or-more pads 102 can also be conveniently placed anywhere along the primary FDL as a result. In addition, being as there are no valves or moving parts, there is a reduced chance of leaking in view of the self-sealing elastomeric tubing of the primary tubing 140.

The secondary FDL can be split at a primary FDL-connecting end of the secondary FDL. The primary FDL-connecting end can include the pair of piercing tips 180. A secondary inlet piercing tip of the pair of piercing tips 180 can be configured to fluidly connect to the primary FDL 103. A secondary outlet piercing tip of the pair of piercing tips 180 can be configured to fluidly connect to the primary FDL 103 in series with the secondary inlet piercing tip as shown in FIGS. 1, 2A, and 2B.

The secondary FDL can be split at a pad-connecting end of the secondary FDL. The pad-connecting end can include a pair of secondary FDL connectors 182. A secondary inlet connector of the pair of secondary FDL connectors 182 can be configured to fluidly connect to the outlet 176 or an outlet connector of the pad including the outlet 176. A secondary outlet connector of the pair of secondary FDL connectors 182 can be configured to fluidly connect to the inlet 174 or an inlet connector of the pad including the inlet 174.

As shown in detail in FIGS. 2A and 2B, when a pad of the one-or-more pads 102 is fluidly connected to the primary FDL 103, the supply fluid can be directed from the primary FDL 103, into the secondary inlet piercing tip of the pair or piercing tips 180 by occluding the folded lumen 142 or the regular lumen 150 with the secondary inlet piercing tip, through the secondary FDL, into the inlet 174 or the inlet connector of the pad to which the secondary outlet connector of the pair of secondary FDL connectors 182 is connected, through the conduit layer 160 of the pad, out the outlet 176 or the outlet connector of the pad to which the secondary inlet connector of the pair of secondary FDL connectors 182 is connected, back through the secondary FDL, out the secondary outlet piercing tip of the pair or piercing tips 180 with occlusion of the folded lumen 142 or regular lumen 150 with the secondary outlet piercing tip, and back into the folded lumen 142 or regular lumen 150 for a subsequent pad.

### Additional Embodiments For Fluidly Connecting Pads

Notwithstanding the foregoing, additional embodiments for fluidly connecting the one-or-more pads 102 to the hydraulic system 108 are set forth below.

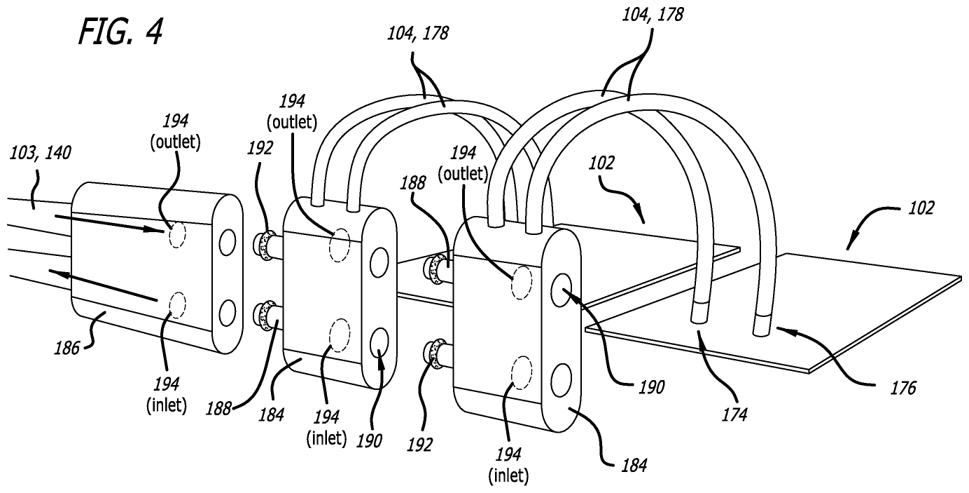

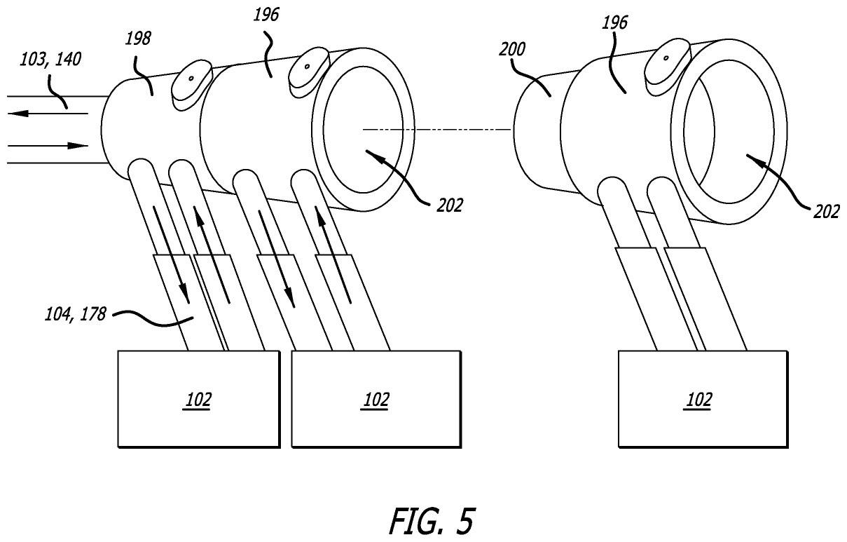

FIGS. 4 and 5 illustrate the one-or-more pads 102 respectively with the one-or-more secondary FDLs 104, wherein each secondary FDL of the one-or-more secondary FDLs 104 includes a secondary FDL plug-and-receptacle connector 184 or 196 configured to fluidly connect to at least a complementary primary FDL connector 186 or 198 of the primary FDL 103 in accordance with some embodiments.

As shown in FIG. 4, the secondary FDL plug-and-receptacle connector 184 is configured to fluidly connect to the complementary primary FDL connector 186 of the primary FDL 103 or an intervening secondary FDL plug-and-receptacle connector of an intervening pad between the secondary FDL plug-and-receptacle connector 184 and the primary FDL connector 186.

The secondary FDL plug-and-receptacle connector 184 includes up to two protrusions 188 extending from a plug portion of the secondary FDL plug-and-receptacle connector 184, wherein the plug portion of the secondary FDL plug-and-receptacle connector 184 is a side of the secondary FDL plug-and-receptacle connector 184 or a portion thereof including a protrusion of the up-to-two protrusions. In addition, the secondary FDL plug-and-receptacle connector 184 includes up to two openings 190 in a receptacle portion of the secondary FDL plug-and-receptacle connector 184, wherein the receptacle portion of the secondary FDL plug-and-receptacle connector 184 is a side of the secondary FDL plug-and-receptacle connector 184 or a portion thereof including an opening of the up-to-two openings 190. Each protrusion of the up-to-two protrusions 188 can extend from the plug portion of the secondary FDL plug-and-receptacle connector 184 in a same direction as another protrusion of the up-to-two protrusions 188 as shown in FIG. 4 by the two protrusions 188 on a same side of the secondary FDL plug-and-receptacle connector 184. Likewise, each opening of the up-to-two openings 190 in the receptacle portion of the secondary FDL plug-and-receptacle connector 184 opens in a same direction as another opening of the up-to-two openings 190 as further shown in FIG. 4 by the two openings 190 on a same side of the secondary FDL plug-and-receptacle connector 184. That said, each protrusion of the up-to-two protrusions 188 can extend from the plug portion of the secondary FDL plug-and-receptacle connector 184 in an opposite direction from another protrusion of the up-to-two protrusions 188. Likewise, each opening of the up-to-two openings 190 in the receptacle portion of the secondary FDL plug-and-receptacle connector 184 can open in an opposite direction from another opening of the up-to-two openings 190. While not shown, the two protrusions 188 are on opposite sides of the secondary FDL plug-and-receptacle connector 184 in such embodiments. Likewise, the two openings 190 are on opposite sides of the secondary FDL plug-and-receptacle connector 184 such that each side of the secondary FDL plug-and-receptacle connector 184 has one protrusion and one opening. Further in such embodiments, each protrusion of the up-to-two protrusions 188 can extend from the plug portion of the secondary FDL plug-and-receptacle connector 184 in a direction of fluid flow for the temperature-controlled fluid. Likewise, each opening of the up-to-two openings 190 in the receptacle portion of the secondary FDL plug-and-receptacle connector 184 can open in an opposite direction of fluid flow for the temperature-controlled fluid.

Each protrusion of the up-to-two protrusions 188 extending from the plug portion of the secondary FDL plug-and-receptacle connector 184 can include at least one circumferential groove including an ‘O’-ring 192 disposed therein. With the ‘O’-ring 192, the protrusion is configured to seal a fluid connection established between the protrusion and a corresponding opening in the receptacle portion of the secondary FDL plug-and-receptacle connector 184.

Each opening of the up-to-two openings 190 in the receptacle portion of the secondary FDL plug-and-receptacle connector 184 can include a valve 194 disposed therein. With the valve 194, the opening is configured to seal off the opening when a corresponding protrusion extending from the plug portion of the secondary FDL plug-and-receptacle connector 184 does not extend through the valve.

As shown in FIG. 5, the secondary FDL plug-and-receptacle connector 196 is also configured to fluidly connect to the primary FDL connector 198 of the primary FDL 103 or an intervening secondary FDL plug-and-receptacle connector of an intervening pad between the secondary FDL plug-and-receptacle connector 196 and the primary FDL connector 198. However, the secondary FDL plug-and-receptacle connector 196 includes a pluggable cap 200 extending from a plug portion of the secondary FDL plug-and-receptacle connector 196, wherein the plug portion of the secondary FDL plug-and-receptacle connector 186 is an end portion of the secondary FDL plug-and-receptacle connector 186 including the cap 200. The secondary FDL plug-and-receptacle connector 196 also includes a socket 202 in a receptacle portion of the secondary FDL plug-and-receptacle connector 196, wherein the receptacle portion of the secondary FDL plug-and-receptacle connector 186 is an end portion of the secondary FDL plug-and-receptacle connector 186 including the socket 202. The cap 200 is configured to insert into the socket 202 of the complementary primary FDL connector 198 of the primary FDL 103 or that of the receptacle portion of another secondary FDL plug-and-receptacle connector. Similarly, the socket 202 is configured to accept insertion of another cap in the plug portion of yet another secondary FDL plug-and-receptacle connector. While not shown, the cap 200, like a protrusion of the up-to-two protrusions 188, can include an ‘O’-ring configured to seal a fluid connection established between the cap 200 and a corresponding socket in a receptacle portion of another secondary FDL plug-and-receptacle connector. And the socket 202, like an opening of the up-to-two openings 190, can include a valve configured to seal off the socket 202 when a corresponding cap extending from the plug portion of another secondary FDL plug-and-receptacle connector does not extend through the valve.

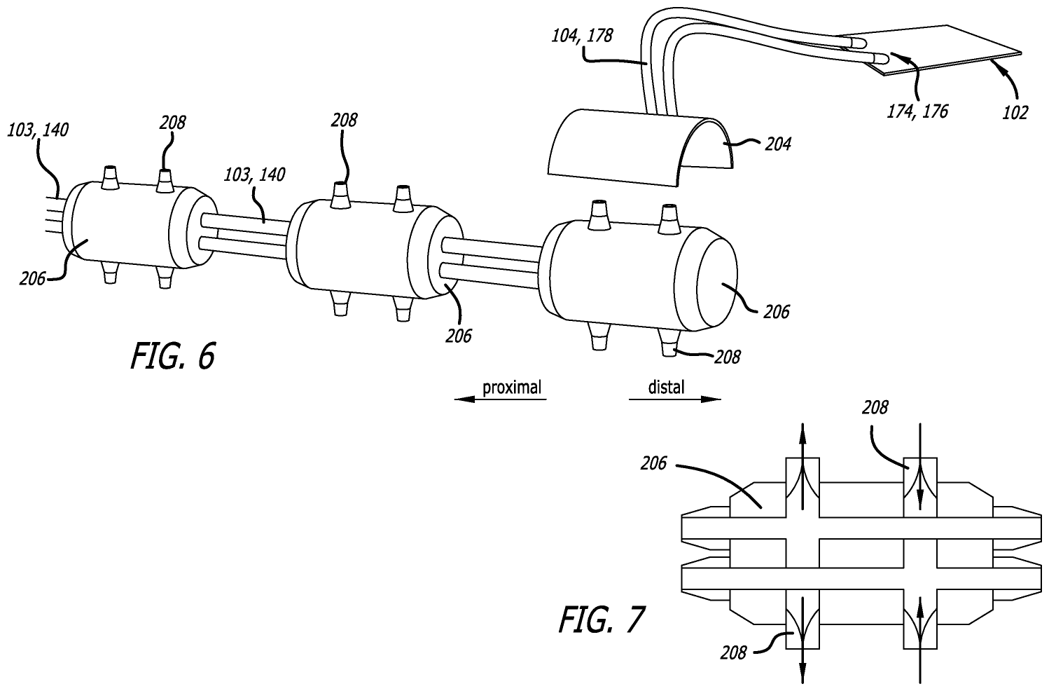

FIG. 6 illustrates a pad of the one-or-more pads 102 including a secondary FDL with a secondary FDL receptacle connector 204 configured to fluidly connect to any primary FDL plug connector of a plurality of primary FDL plug connectors 206 along a length of the primary tubing 140 of the primary FDL 103 in accordance with some embodiments. FIG. 7 illustrates a longitudinal cross section of a primary FDL plug connector of the plurality of primary FDL plug connectors 206 of the primary FDL 103 in accordance with some embodiments.

As shown, the primary FDL 103 can include the plurality of primary FDL plug connectors 206 regularly spaced along the length of the primary tubing 140. While each primary FDL plug connector of the plurality of primary plug FDL connectors 206 is shown regularly spaced from an adjacent primary FDL plug connector, groups such as pairs of the primary FDL plug connectors 206 can also be regularly spaced along the length of the primary tubing 140. That said, the plurality of primary FDL plug connectors 206 can be spaced differently along the length of the primary tubing 140 such as randomly along the length of the primary tubing 140, as needed.

Each primary FDL plug connector of the plurality of primary FDL plug connectors 206 is configured to fluidly connect up to two secondary FDL receptacle connectors 204 respectively of two pads. That said, each primary FDL plug connector of the plurality of primary FDL plug connectors 206 can be configured to fluidly connect up to four secondary FDL receptacle connectors 204 respectively of four pads orthogonally around the primary FDL plug connector. Continuing with the illustrated embodiment, however, each primary FDL plug connector of the plurality of primary FDL plug connectors 206 includes a pair of oppositely oriented plug portions of the primary FDL plug connector. Each plug portion of the pair plug portions includes a pair of protrusions 208 extending therefrom, wherein a proximal protrusion of each pair of protrusions 208 is an outlet configured for discharging the supply fluid therefrom and a distal protrusion of each pair of protrusions 208 is an inlet configured for receiving the return fluid.

Again, the secondary FDL receptacle connector 204 is configured to fluidly connect to any primary FDL plug connector of the plurality of primary FDL plug connectors 206 along the length of the primary tubing 140. As a complement to each primary FDL plug connector of the plurality of primary FDL plug connectors 206, the secondary FDL receptacle connector 204 includes a pair of openings (not shown) in a receptacle portion of the secondary FDL receptacle connector 204 configured to form fluid connections with the pair of protrusions 208. While the secondary FDL receptacle connector 204 can be configured to enforce a particular orientation such as that shown in FIG. 6 for fluidly connecting to a primary FDL plug connector of the plurality of primary FDL plug connectors 206, the secondary FDL receptacle connector 204 and the pad to which it is indirectly connected can be configured to allow either of the two discernable orientations shown in FIG. 6. Like the alternative embodiment set forth above with respect to the secondary FDL plug-and-receptacle connector 184 of FIG. 4, however, each of the primary FDL plug connector and the secondary FDL receptacle connector 204 can be modified to include a protrusion and an opening, wherein the foregoing protrusions extend from their respective connectors in a direction of fluid flow for the temperature-controlled fluid and the foregoing openings of their respective connectors open in an opposite direction of fluid flow for the temperature-controlled fluid, thereby further enforcing a particular connection orientation.

### Methods

Methods of the systems and pads include methods of use. For example, a method of using the system 100 can include one or more steps selected from a control module-connecting step, a backing-removal step, a pad-placing step, a pad-connecting step, a piercing step, a fluid-conveying step, a fluid-circulating step, and a disconnecting step.

The control module-connecting step can include fluidly connecting the primary FDL connector 148 of the connector end 146 of the primary FDL 103 to the control-module connector of the control module 101. As set, forth above, the primary tubing 140 can include the longitudinal septum 149 short of the closed end 144 of the primary FDL 103 that forms the folded lumen 142 in the primary tubing 140. The folded lumen 142 can be configured for conveying the supply fluid from the inlet of the primary FDL connector 148. The folded lumen 142 can also be configured for conveying the return fluid to the outlet of the primary FDL connector 148.

As an alternative to the foregoing control module-connecting step, the control module-connecting step can include fluidly connecting the pair of primary FDL connectors 154 respectively at opposing connector ends of the primary FDL 103 to the pair of control-module connectors of the control module 101. As set forth above, the primary tubing 140 can include the regular lumen 150. The regular lumen 150 can be configured for conveying the supply fluid from the inlet of the primary inlet connector of the pair of primary FDL connectors 154. The regular lumen 150 can also be configured for conveying the return fluid to the outlet of the primary outlet connector of the pair of primary FDL connectors 154.

The backing-removal step can include removing the backing 158 of a pad of the one-or-more pads 102 to reveal the adhesive layer 164 before the pad-placing step. As set forth above, the backing 158 is configured to maintain integrity of at least the adhesive layer 164 prior to using the pad.

The pad-placing step includes placing the pad on a portion of a patient's body with the adhesive layer 164 of the pad in contact with skin S (see FIG. 8) of the portion of the patient's body.

If a secondary FDL of the one-or-more secondary FDLs 104 is not connected to the foregoing pad of the one-or-more pads 102, the method can further include a pad-connecting step. The pad-connecting step can include fluidly connecting the secondary outlet connector of the pair of secondary FDL connectors 182 at the split pad-connecting end of the secondary FDL to the outlet 176 or the outlet connector of the pad including the outlet 176. The pad-connecting step can also include fluidly connecting the secondary inlet connector of the pair of secondary FDL connectors 182 to the inlet 174 or the inlet connector of the pad including the inlet 174.

The piercing step can include piercing the primary tubing 140 of the primary FDL 103 with the pair of piercing tips 180 of the secondary FDL to fluidly connect the secondary FDL to the primary FDL 103.

The fluid-conveying step includes conveying a temperature-controlled fluid as the supply fluid to the pad. The supply fluid is provided by the hydraulic system 108 of the control module 101 by way of the primary FDL 103 and the secondary FDL.

The fluid-circulating step includes circulating the supply fluid through the conduit layer 160 of the pad body 156 to cool or warm the portion of the patient's body as needed in accordance with TTM.

The fluid-circulating step includes transferring heat between the supply fluid and the portion of the patient's body by thermal conduction through the adhesive layer 164. For example, the fluid-circulating step can include circulating a cool fluid through the conduit layer 160 to bring the patient into hypothermia. The fluid-circulating step can include circulating a warm fluid through the conduit layer 160 to bring the patient into normothermia.

The disconnecting step can include removing the pair of piercing tips 180 of the secondary FDL from the primary tubing 140 of the primary FDL 103 to disconnect the secondary FDL tubing from the primary FDL 103. As set forth above, the primary tubing 140 can be self-sealing elastomeric tubing configured to prevent leaking when removing the pair of piercing tips 180 of the secondary FDL from the primary tubing 140 of the primary FDL 103.

While the method set forth above is described with reference to a single pad of the one-or-more pad 102, it should be understood that any number of pads of the one-or-more pads 102 can be used as necessary to effectuate a desired treatment by way of TTM.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A system for targeted temperature management (“TTM”), comprising:
a control module including a hydraulic system configured to provide a temperature-controlled fluid;
a primary fluid delivery line (“FDL”) including primary tubing configured to convey the temperature-controlled fluid as a supply fluid from the hydraulic system and convey a return fluid back to the hydraulic system; and
one or more pads configured for placement on one or more portions of a patient's body, respectively, each pad of the one-or-more pads including:
a multilayered pad body including a conduit layer including one or more conduits configured to convey the supply fluid through the conduit layer; and
a secondary FDL including secondary tubing configured to convey the supply fluid from the primary FDL and convey the return fluid back to the primary FDL, the secondary FDL including a pair of piercing tips configured to pierce the primary tubing and, thereby, fluidly connect the secondary FDL to the primary FDL.

2. The system of claim 1, wherein the primary tubing is self-sealing elastomeric tubing.

3. The system of claim 1, wherein the primary tubing includes a closed end and a connector end including a primary FDL connector configured to fluidly connect to a control-module connector of the control module.

4. The system of claim 3, wherein the primary tubing includes a longitudinal septum short of the closed end of the primary tubing, the longitudinal septum forming a folded lumen in the primary tubing configured to convey the supply fluid from an inlet of the primary FDL connector to the closed end of the primary tubing and convey the return fluid from the closed end to an outlet of the primary FDL connector of the primary tubing.

5. The system of claim 1, wherein the primary tubing includes a pair of opposing connector ends respectively including a pair of primary FDL connectors configured to fluidly connect to a pair of control-module connectors of the control module.

6. The system of claim 5, wherein the primary tubing includes a regular lumen in the primary tubing configured to convey the supply fluid from an inlet of a primary inlet connector of the pair of primary FDL connectors and convey the return fluid to an outlet of a primary outlet connector of the pair of primary FDL connectors.

7. The system of claim 1, the pad body further including:
an impermeable film over the conduit layer configured to retain the supply fluid in the conduit layer; and
a thermally conductive adhesive layer over the impermeable film configured for placement on a portion of a patient's body.

8. The system of claim 7, wherein the adhesive layer includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

9. The system of claim 7, each pad of the one-or-more pads further including a backing over the adhesive layer in a ready-to-use state of the pad, the backing configured to maintain integrity of at least the adhesive layer prior to use of the pad.

10. The system of claim 1, each pad of the one-or-more pads further comprising:
a pad inlet configured for charging the conduit layer with the supply fluid; and
a pad outlet configured for discharging the return fluid from the conduit layer.

11. The system of claim 10, wherein the secondary FDL is split at a pad-connecting end of the secondary FDL, the pad-connecting end including a pair of secondary FDL connectors including a secondary inlet connector configured to fluidly connect to the pad outlet and a secondary outlet connector configured to fluidly connect to the pad inlet.

12. The system of claim 1, wherein the secondary FDL is split at a primary FDL-connecting end of the secondary FDL, the primary FDL-connecting end including the pair of piercing tips having a secondary inlet piercing tip configured to fluidly connect to the primary FDL and a secondary outlet piercing tip configured to fluidly connect to the primary FDL in series with the secondary inlet piercing tip.

13. The system of claim 1, the hydraulic system further including:
a chiller evaporator configured for fluid cooling;
a heater configured for fluid heating, the chiller evaporator and the heater, together, configured to provide the temperature-controlled fluid;
a hydraulic-system outlet configured for discharging the supply fluid from the hydraulic system; and
a hydraulic-system inlet configured for charging the hydraulic system with the return fluid to continue to produce the temperature-controlled fluid.

14. The system of claim 1, the control module further including one or more processors, primary memory, and instructions stored in the primary memory configured to instantiate one or more processes for TTM with the control module, at least one process of the one-or-more processes being a fluid delivery-adjustment process configured for determining and adjusting parameters of fluid delivery in accordance with a number of FDL-connected pads of the one-or-more pads.

15. A method of a system for targeted temperature management (“TTM”), comprising:
piercing primary tubing of a primary fluid delivery line (“FDL”) with a pair of piercing tips of a secondary FDL to fluidly connect the secondary FDL to the primary FDL;
placing a pad on a portion of a patient's body with a thermally conductive adhesive layer of a multilayered pad body of the pad in contact with skin of the portion of the patient's body;
conveying a temperature-controlled fluid as a supply fluid to the pad, the supply fluid from a hydraulic system of a control module by way of the primary and secondary FDLs; and
circulating the supply fluid through a conduit layer of the pad body to cool or warm the portion of the patient's body as needed in accordance with TTM.

16. The method of claim 15, further comprising fluidly connecting a primary FDL connector of a connector end of the primary FDL to a control-module connector of the control module, the primary tubing including a longitudinal septum short of a closed end of the primary FDL that forms a folded lumen in the primary tubing for conveying the supply fluid from an inlet of the primary FDL connector and conveying a return fluid to an outlet of the primary FDL connector.

17. The method of claim 15, further comprising fluidly connecting a pair of primary FDL connectors respectively at opposing connector ends of the primary FDL to a pair of control-module connectors of the control module, the primary tubing including a regular lumen for conveying the supply fluid from an inlet of a primary inlet connector of the pair of primary FDL connectors and conveying a return fluid to an outlet of a primary outlet connector of the pair of primary FDL connectors.

18. The method of claim 15, further comprising:
fluidly connecting a secondary outlet connector of a pair of secondary FDL connectors at a split pad-connecting end of the secondary FDL to a pad outlet of the pad; and
fluidly connecting a secondary inlet connector of the pair of secondary FDL connectors to a pad inlet of the pad.

19. The method of claim 15, further comprising removing a backing of the pad to reveal the adhesive layer before placing the pad on the portion of the patient's body, the backing configured to maintain integrity of at least the adhesive layer prior to using the pad.

20. The method of claim 15, wherein circulating the supply fluid through the conduit layer transfers heat between the supply fluid and the portion of the patient's body by thermal conduction through the adhesive layer, the adhesive layer including a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

21. The method of claim 15, wherein circulating the supply fluid through the conduit layer includes circulating a cool fluid through the conduit layer to bring the patient into hypothermia.

22. The method of claim 15, wherein circulating the supply fluid through the conduit layer includes circulating a warm fluid through the conduit layer to bring the patient into normothermia.

23. The method of claim 15, further comprising removing the pair of piercing tips of the secondary FDL from the primary tubing of the primary FDL to disconnect the secondary FDL tubing from the primary FDL, the primary tubing being self-sealing elastomeric tubing configured to prevent leaking when removing the pair of piercing tips of the secondary FDL from the primary tubing of the primary FDL.

24-36. (canceled)
